(19) United States
(12) Patent Application Publication
(10) Pub. No.: US 2016/0345818 A1
(43) Pub. Date: Dec. 1, 2016

(54) EYEBLINK MEASUREMENT METHOD, EYEBLINK MEASUREMENT APPARATUS, AND NON-TRANSITORY COMPUTER-READABLE MEDIUM

(71) Applicant: HAMAMATSU PHOTONICS K.K., Hamamatsu-shi (JP)

(72) Inventors: Kazutaka SUZUKI, Hamamatsu-shi (JP); Munenori TAKUMI, Hamamatsu-shi (JP); Naotoshi HAKAMATA, Hamamatsu-shi (JP); Haruyoshi TOYODA, Hamamatsu-shi (JP); Yoshinori MATSUI, Hamamatsu-shi (JP)

(21) Appl. No.: 15/163,921

(22) Filed: May 25, 2016

(30) Foreign Application Priority Data
May 28, 2015 (JP) 2015-108365

(51) Int. Cl. A61B 3/00; A61B 5/18; A61B 3/113

(52) U.S. Cl. CPC A61B 3/0025; A61B 3/113; A61B 5/18

(57) ABSTRACT

An eyeblink measurement method includes a step of detecting reflected light from a part including a target person's eyelid and eye and outputting an image signal of the reflected light, using a photodetector, a step of calculating a position of a corneal reflected light produced on the eye in the part and a position of the eyelid in the part based on the image signal, a step of correcting the position of the eyelid based on the position of the corneal reflected light, and a step of calculating a feature amount regarding blinking based on a temporal change in the corrected position of the eyelid.

### TECHNICAL FIELD

An aspect of the present invention relates to an eyeblink measurement method, an eyeblink measurement apparatus, and an eyeblink measurement program for measuring a target person's blinking.

### BACKGROUND

Conventionally, an apparatus that detects blinking using a video camera to quantify drowsiness has been developed in order to detect a driver's drowsiness and the like. For example, the apparatus described in the following patent document 1 searches a face image obtained by a CCD camera for an eye region to identify an extraction position, extracts a small-region image including an eye as an eye image based on the identified extraction position, and measures an eyelid opening degree based on the eye image.

Patent Document 1: Japanese Unexamined Patent Publication No. H7-313459

### SUMMARY

However, according to the apparatus described in the above patent document 1, because it becomes necessary when a plurality of face images are successively obtained to extract the eye regions successively, there is a limit to performing a high-speed detection processing. It is thus difficult to accurately obtain an evaluation value regarding a target person's blinking motion in real time.

Therefore, the present invention has been made in view of such problems, and it is an object of the present invention to provide an eyeblink measurement method and an eyeblink measurement apparatus capable of obtaining an evaluation value regarding a target person's blinking motion at a high speed and high accuracy.

In order to solve the above-mentioned problems, an eyeblink measurement method according to a mode of the present invention includes a step of detecting light from a part including a target person's eyelid and eye and outputting a detection signal of the light, using a photodetector, a step of calculating a position of a corneal reflected light produced on the eye in the part and a position of the eyelid in the part based on the detection signal, a step of correcting the position of the eyelid based on the position of the corneal reflected light, and a step of calculating a feature amount regarding blinking based on a temporal change in the corrected position of the eyelid.

Or an eyeblink measurement apparatus according to another mode of the present invention includes a photodetector configured to detect light from a part including a target person's eyelid and eye and output a detection signal, and a processor electrically coupled to the photodetector and configured to input the detection signal. The processor is configured to calculate a position of a corneal reflected light produced on the eye in the part and a position of the eyelid in the part based on the detection signal, correct the position of the eyelid based on the position of the corneal reflected light, and calculate a feature amount regarding blinking based on a temporal change in the corrected position of the eyelid.

Or non-transitory computer-readable medium according to another mode of the present invention contains program instructions for causing a processor to perform the method of, in an eyeblink measurement apparatus that measures eye blinking of a target person using an image of a part including the target person's eyelid and eye, calculating a position of a corneal reflected light produced on the eye in the part and a position of the eyelid in the part based on the image, correcting the position of the eyelid based on the position of the corneal reflected light, and calculating a feature amount regarding blinking based on a temporal change in the corrected position of the eyelid.

By the eyeblink measurement method, eyeblink measurement apparatus, and the non-transitory computer-readable medium containing program of the above-mentioned modes, a detection signal (image) of light from the part including the target person's eyelids and eye is generated, a corneal reflected light position and an eyelid position in said part are calculated based on the detection signal (image), and then the eyelid position is corrected based on the corneal reflected light position, and from a temporal change in the corrected eyelid position, a feature amount regarding blinking is calculated. This allows calculating a temporal change in eyelid position corresponding to a movement of the eyelid itself by a simple calculation even when the relative position of the target person's eye with respect to the apparatus changes. As a result, a feature amount regarding blinking can be obtained at a high speed and high accuracy from the temporal change in the target person's eyelid position.

### BRIEF DESCRIPTION OF THE DRAWINGS

FIG. 1 is a block diagram showing a schematic configuration of an eyeblink measurement system 1 according to a preferred embodiment of the present invention.

FIGS. 2A, 2B and 2C include views showing calculation images of an upper eyelid position by the position calculating unit 11 in FIG. 1.

FIG. 3 is a graph showing an example of temporal changes in the eyelid position calculated by the position calculating unit 11 in FIG. 1 and temporal changes in the corneal reflected light position calculated by the movement amount calculating unit 13 in FIG. 1

FIGS. 4A, 4B and 4C include views showing images of image signals that are obtained at timing corresponding to times T1, T2, and T3 shown in FIG. 3.

FIG. 5 is a graph showing an example of temporal changes in the eyelid position calculated by the position correcting unit 15 in FIG. 1.

FIG. 6 is a flowchart showing a procedure of operation to calculate an eye blink feature amount by the eyeblink measurement system 1 in FIG. 1.

### DETAILED DESCRIPTION

Hereinafter, a preferred embodiment of an eyeblink measurement apparatus, an eyeblink measurement method, and an eyeblink measurement program according to the present invention will be described in detail with reference to the drawings. In addition, the same or corresponding parts will be denoted by the same reference signs in the description of the drawings, and overlapping description will be omitted.

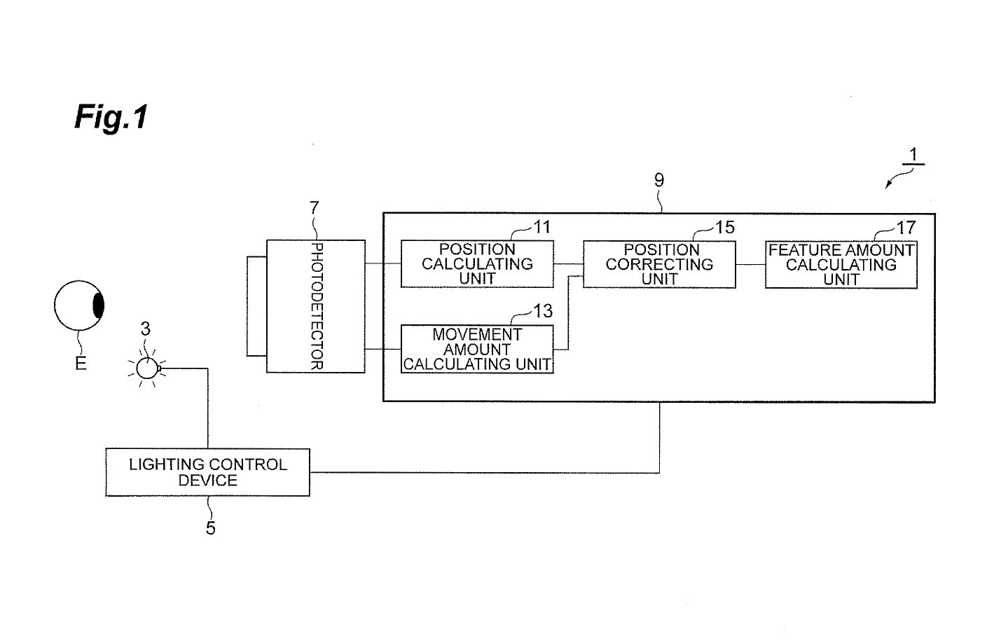

FIG. 1 is a block diagram showing a schematic configuration of an eyeblink measurement system 1 according to a preferred embodiment of the present invention. The eyeblink measurement system 1 shown in FIG. 1 is constructed so as to detect light from a subject's (target person's) eye E temporally in succession at a predetermined frame rate and output an evaluation value regarding the subject's eye blinking in a quantified manner. This eyeblink measurement system 1 includes a light source 3 that irradiates illumination light onto the target person's eye E, a lighting control device 5 that controls the light source 3, a photodetector 7 that detects light from the eye E, and a processor 9 that processes a detection signal output from the photodetector 7. Here, the eyeblink measurement system 1 may be constructed so as to be able to measure the subject's two right and left eyes simultaneously, or may be constructed so as to be able to measure the subject's two right and left eyes separately. In the case of the construction for measuring the right and left eyes simultaneously, two photodetectors 7 may be provided for detection of the respective eyes, or there may be a single photodetector 7 that detects the two eyes simultaneously so that a detection signal is processed in the processor 9 in a manner separated into signals corresponding to the two eyes.

The light source 3 is a lighting means that illuminates the subject's eye E inclusive of its periphery including the upper and lower eyelids, and is suitably constructed by, for example, an LED that generates infrared light. By the light source 3 irradiating the eye E and their periphery with infrared light, the infrared light is reflected in the eye E and their periphery to produce an optical image. Also, the irradiation by the light source 3 can also produce a corneal reflected light on the eye E. The light source 3 may be constructed so as to be able to irradiate an illumination light for lighting an eye area including the eye E and its periphery and an illumination light for generating corneal reflected light separately, or may be constructed so as to be able to irradiate these simultaneously. The separately irradiating construction is preferred because adjusting an illumination light for generating corneal reflected light makes it possible to adjust the illuminance, shape, and size of a corneal reflected light. In this case, adjustment is performed by a lighting control device 5 to be described later so that, before or during a measurement of the subject's eyelid movement, the luminance distribution of corneal reflected light approximates a Gaussian distribution, the illuminance thereof comes in a range to be within the width of a dynamic range of the photodetector 7, and the maximum luminance thereof does not exceed the dynamic range of the photodetector 7. Also, when the separately irradiating construction is adopted, control is performed by the lighting control device 5 so that an illumination light for eye area lighting and an illumination light for generating corneal reflected light are alternately lit, a signal indicating their lighting timing is output to the processor 9, and detection signals at the lighting timing of the two illumination lights output from the photodetector 7 are separately processed in the processor 9. On the other hand, the simultaneously irradiating construction is made so as to be able to irradiate an illumination light for eye area lighting and an illumination light for generating corneal reflected light at different wavelengths, which is preferred. In this case, at the time of detection of light in the photodetector 7 or at the time of analysis of a detection signal in the processor 9, reflected lights due to both illumination lights are distinguished according to the wavelength and detection signals responding to the respective reflected lights are separately processed. This light source 3 is disposed at a position where irradiating the subject from below is possible in order to enable observing a corneal reflected light without receiving the influence of eyelashes on the lower eyelid or of the upper eyelid. The position of a corneal reflected light to be generated is accordingly at a lower side relative to the pupillary area, so that the observing time of corneal reflected light at the time of eye blinking can be prolonged, and the corneal reflected light can be stably measured even for a subject having symptoms of blepharoptosis or the like.

In addition, the light source 3 is not limited to an infrared LED, and other types of light sources can be used. For example, it may be a light source that emits near infrared light, may be a construction for which a film that passes infrared light or near infrared light is combined with a lamp light source, or may be a construction that uses a laser light satisfying safety standards as direct light or indirect light. Also, as a construction for realizing an appropriate illuminance, besides a construction using a plurality of lighting at the same time, a construction with a lens embedded in a light emitting unit may be used to suppress illumination light from scattering so as to effectively illuminate a desired region. Also, a construction may be adopted that shapes laser light energy into a desired lighting shape using a spatial modulation device to perform effective eye area lighting.

The lighting control device 5 is a control unit that controls the light amount of the light source 3 so as to illuminate the subject's eye E at a predetermined brightness. The lighting control device 5 also adjusts and controls the light amount and emission wavelength of the light source 3 so that a light amount, illumination light wavelength, and reflected light shape suitable for eyelid position detection or corneal reflected light detection of the subject's eye E can be obtained. Further, the lighting control device 5 is electrically connected with the processor 9, and through synchronous control by the processor 9, controls the light emission timing to the subject's eye E. Moreover, the lighting control device 5 may perform control so as to make illumination light of the light source 3 blink before the start of measurement in order to make it possible to determine by the processor 9 or the photodetector 7 that the subject's eye E is within a measurement range of the photodetector 7 or in order to realize an alignment function for the subject's eye E by the processor 9 or the photodetector 7.

The photodetector 7 is an imaging device that detects a reflected light from the part of the eye E including the upper eyelid and lower eyelid at a predetermined frame rate to generate and output a two-dimensional image signal (detection signal). As such an imaging device, a vision camera having a vision chip that performs image acquisition to image processing can be mentioned. This photodetector 7 has specifications or settings (e.g. wavelength sensitivity, light amount sensitivity, angle of view of lens, magnification of lens, frame rate, etc.) optimal for detection of the subject's eyelid movement. For example, the photodetector 7 is preferably a construction that is capable of photographing at a frame rate higher than that of a common video camera. Because an eyelid motion at the time of eye blinking is performed in about 200 msec, an outline of eyelid motion behaviors can be captured if the frame rate is on the order of 10 Hz, but a construction having a frame rate of 60 Hz or more is used in order to make a minute eyelid motion or an irregular motion at the time of eyelid closure detectable. Also, the angle of view of a lens is set so that an eyelid movement of less than 1 mm can be captured. For example, the angle of view is set, in consideration of the number of pixels of the photodetector 7, the brightness of the eye area judged from a composite element of the exposure time, the dynamic range, the illuminance of illumination light, the brightness of the lens, etc., and the size of a save area of data of an image signal worked out from these parameters, so that the whole eye area is contained in the longitudinal direction of a screen of the image signal. Also, when a camera having a high resolution is used as the photodetector 7, the angle of view may be set to a wide angle, and only a necessary region may be clipped to be saved or analyzed in the photodetector 7 or the processor 9.

Here, as the photodetector 7, a construction other than a video camera or a vision camera may be used. For example, as the photodetector 7, a simpler sensor such as a photodiode, a photodetector, a linear image sensor, a profile sensor, or an area image sensor may be used, besides a sensor that detects the position of a bright spot to output position information such as a profile sensor. When the detection content is eye blinking and when a video camera is used as the photodetector 7, an eyelid position extraction processing using an image processing technique such as edge extraction, Hough transform, or binarization or a processing for determining an eyelid position from a luminance profile calculated from an image signal, etc., is performed. Alternatively, as the photodetector 7, a construction may be adopted that includes a lighting unit (e.g. a laser array, an LED array, or the like) that irradiates/projects a marker in a dotted line-like, line-like, or band-like shape or a shape similar thereto onto the eye area and a detection unit (e.g. a profile sensor, a photodiode, a photodetector, a linear image sensor, an area sensor, or the like) that extracts an eyelid position by capturing reflection on an eye surface without capturing a scattered light on skin.

The processor 9 is an image processing processor incorporated with a CPU that processes an image signal output from the photodetector 7 and a memory such as a RAM and a ROM. The processor 9 is included in a computer. For example, the computer is a personal computer or a smart device represented by smartphone or a tablet type computer, or the like. This processor 9 includes as its functional components a position calculating unit 11, a movement amount calculating unit (position calculating unit) 13, a position correcting unit 15, and a feature amount calculating unit 17. The position calculating unit 11, the movement amount calculating unit 13, the position correcting unit 15, and the feature amount calculating unit 17 may be realized by hardware in the processor 9, or may be realized by software (an eyeblink measurement program) stored in the processor 9. Also, a part or the whole of these functional units may be provided in the photodetector 7. Also, the functions of the position calculating unit 11, the movement amount calculating unit 13, the position correcting unit 15, and the feature amount calculating unit 17 may be realized by the same processor, or may be realized by different processors. A program that makes the processor 9 function as the position calculating unit 11, the movement amount calculating unit 13, the position correcting unit 15, and the feature amount calculating unit 17 may be stored in a storage device (non-transitory computer-readable medium) within the processor 9, or may be stored in a non-transitory computer-readable medium that is electrically connected with the processor 9.

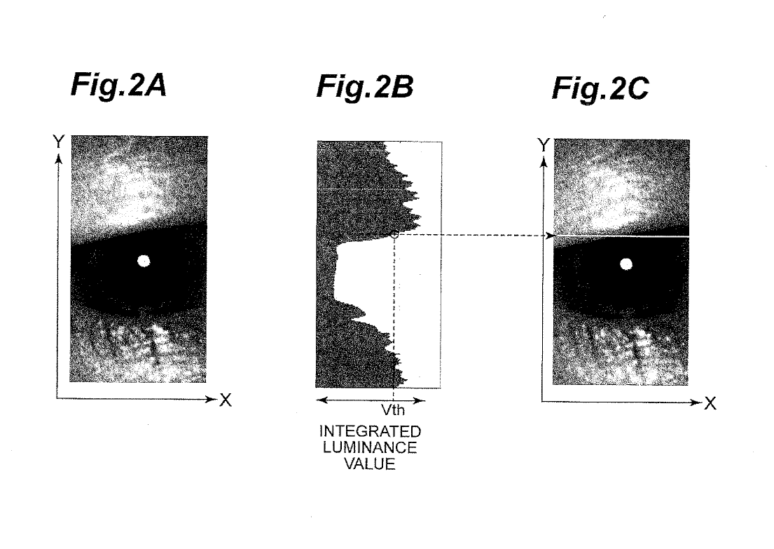

The position calculating unit 11 of the processor 9 calculates positions of the upper eyelid and lower eyelid of the subject's eye E based on an image signal output from the photodetector 7. The position calculating unit 11, by processing for a plurality of image signals output at a predetermined frame rate from the photodetector 7 as a target, calculates a temporal change in the position of the upper eyelid and lower eyelid. The position of the upper eyelid and lower eyelid is calculated by an image processing such as edge extraction or Hough transform targeting an image signal or a processing for determining an eyelid position from a luminance profile calculated from an image signal (refer to Japanese Unexamined Patent Publication No. 2012-085691). FIGS. 2A, 2B and 2C show calculation images of an upper eyelid position by the position calculating unit 11, in which FIG. 2A shows an example of an image signal as a processing target, FIG. 2B shows an example of a luminance profile calculated based on the image signal in FIG. 2A as a target, and FIG. 2C shows an upper eyelid position calculated based on the image signal in FIG. 2A as a target. The position calculating unit 11 thus performs, for the image signal (FIG. 2A) having pixels arrayed two-dimensionally at every x-coordinate corresponding to horizontal positions and every y-coordinate corresponding to vertical positions as a target, integration of x-coordinate pixels in the image signal for each of the y-coordinates to thereby calculate a luminance profile (FIG. 2B). The position calculating unit 11 then calculates a vertical position on a y-coordinate where an integrated luminance value in the luminance profile reaches a threshold Vth or more as the upper eyelid position. This threshold Vth may be set from a luminance value of upper eyelid position coordinates detected by image processing, besides being automatically adjusted in value in an adaptive manner from the sum total of luminance values. Also, the position calculating unit 11 calculates a lower eyelid position in the same manner.

The movement amount calculating unit 13 of the processor 9 calculates a position of a corneal reflected light produced on the subject's eye E based on an image signal output from the photodetector 7. By using properties of corneal reflected light that this is brighter than a skin image or wrinkle image due to a scattered light from a skin surface and the fluctuation in the signal intensity of corneal reflected light is also small, the movement amount calculating unit 13 calculates a corneal reflected light position on two-dimensional coordinates (x-coordinate and y-coordinate) in the image signal by a centroid calculation targeting the image signal. Further, the movement amount calculating unit 13, by processing for a plurality of image signals output at a predetermined frame rate from the photodetector 7 as a target, calculates a temporal change in corneal reflected light position, and obtains the temporal change in corneal reflected light position as a movement amount of position with respect to the photodetector 7 of the subject's eye area.

Here, in a situation where a corneal reflected light disappears due to the subject's eyeblink motion, the movement amount calculating unit 13 performs processing as follows. Concretely, for a region detected as a corneal reflected light as a target, the movement amount calculating unit 13 calculates an elliptical feature amount such as a ratio of the long axis and the short axis or a moment value, and determines the truth or falsity of the detected corneal reflected light based on whether the elliptical feature amount has become a threshold or more. As other methods, based on whether the light amount of a corneal reflected light region has changed to a threshold or more, the truth or falsity of the corneal reflected light may be determined based on a total number of bright spots of a threshold or more on the image signal or its increase or decrease, or the truth or falsity thereof may be determined based on the area of a set of adjacent bright spots. This allows an improvement in the degree of accuracy of a detection position of corneal reflected light against not only the disappearance of the corneal reflected light due to the eyelid covering the cornea during an eyeblink motion but also diffused reflection by skin or makeup.

The position correcting unit 15 of the processor 9 corrects an eyelid position calculated by the position calculating unit 11 based on a movement amount of the eye area position calculated by the movement amount calculating unit 13. Concretely, for a temporal change in eyelid position as a target, correction is performed in a manner of subtracting, from an eyelid position at each of the times, the portion of a movement amount of the eye area position corresponding to that time. By doing so, the portion of a movement of the subject's eye area can be cancelled out in a calculated change in eyelid position, so that a temporal change in eyelid position that reflects a movement of the eyelid itself can be determined. Here, at timing where a real corneal reflected light position has not been obtained, the position correcting unit 15 may correct the eyelid position according to a position change of another eye area feature amount such as an eyeball position.

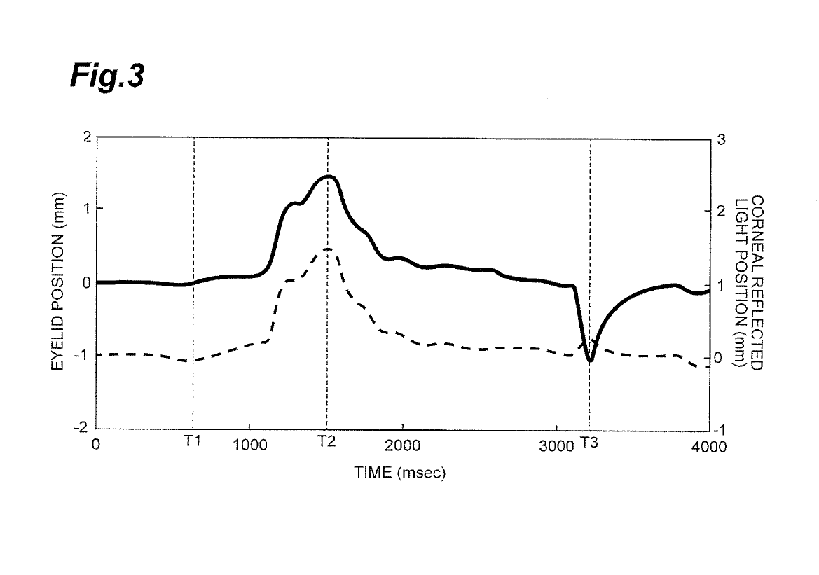

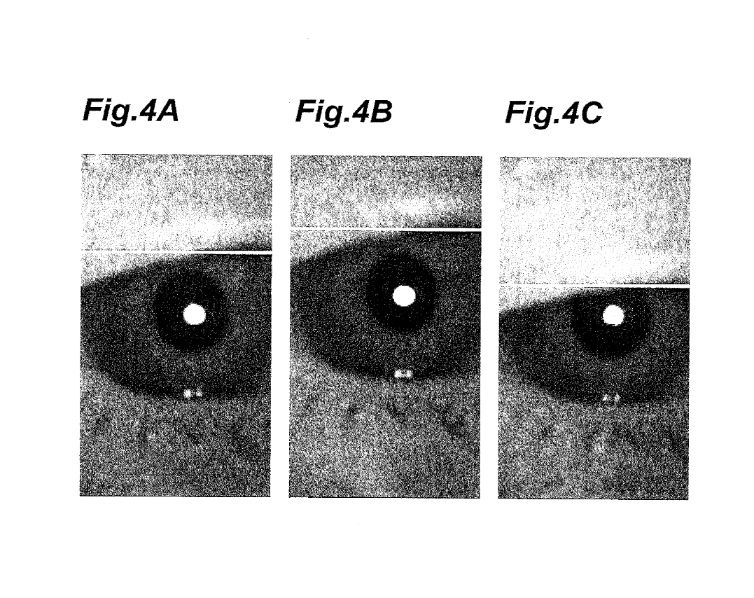

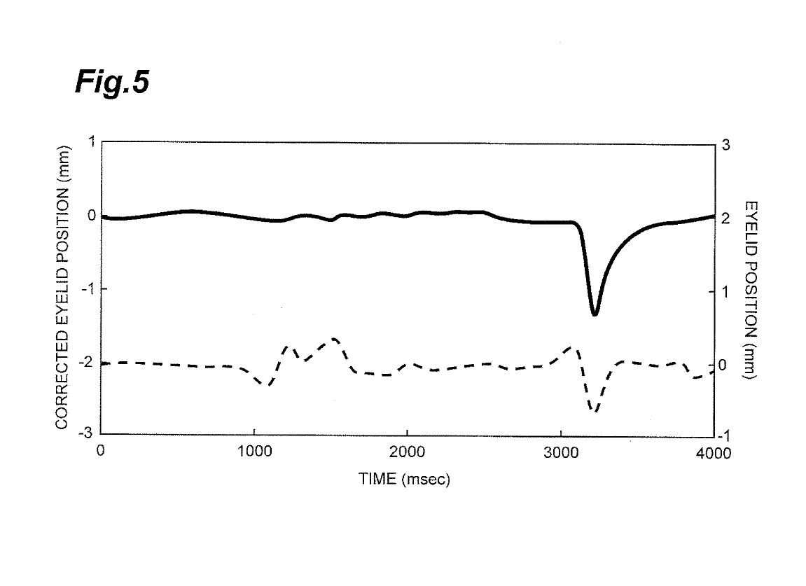

FIG. 3 is a graph showing an example of temporal changes in the vertical position of the eyelid calculated by the position calculating unit 11 and temporal changes in the vertical position of corneal reflected light calculated by the movement amount calculating unit 13. The temporal changes in eyelid position are shown by a solid line, and the temporal changes in corneal reflected light position are shown by a dotted line. FIG. 4A, FIG. 4B, and FIG. 4C show image signals that are obtained at timing corresponding to times T1, T2, and T3 shown in FIG. 3, respectively. FIG. 5 is a graph showing temporal changes in the vertical position of the eyelid corrected by the position correcting unit 15 based on the data shown in FIG. 3. As shown in these figures, a state of the eye area moving upward from time T1 to time T2 and then the eye area returning to the original position to time T3 is detected in an image signal, and changes in corneal reflected light position are also calculated correspondingly thereto (dotted line in FIG. 3). On the other hand, the eyelid position calculated reflects an eyelid movement of approximately 1 mm at time T3 and also reflects changes accompanying eye area movement (solid line in FIG. 3). The movement in eyelid position at time T2 and the movement in eyelid position at time T3 are both detected as changes in eyelid position, but an eyelid movement actually performed by the subject is only the movement at time T3. In the case of a system without a correction function of the eyelid position, it is difficult to distinguish between various patterns of the subject's body movement that are different in amplitude, period, and speed and blinking motion that vary in amplitude, period, and speed, and as a result, a signal that is not of a blinking motion tends to be included as a noise component. In contrast, the position correcting unit 15 allows obtaining an eyelid position from which the influence of an eye area movement caused by the subject's body movement and the like has been removed and which reflects a movement of the eyelid itself (solid line in FIG. 5). In addition, the position of corneal reflected light at time T3 moves upward (dotted line in FIG. 3). This is because of a change in gazing direction during eye blinking. Although a change in gazing direction and body movement cannot be separated from changes in corneal reflected light position, because a position change of corneal reflected light due to a gaze shift is slight, there is little effect on positional accuracy even when a position change of corneal reflected light is subtracted from a temporal change in eyelid position. When it is desired to minimize the effect on accuracy, the position correcting unit 15 may extract an eyeblink motion period from time-dependent changes in the eyelid position after correction, and perform a recalculation so as not to apply a correction based on a temporal change in corneal reflected light position for said period. Discontinuity that occurs at the boundary between the periods for which the correction was carried out and was not carried out is eliminated, when determining time-dependent changes in eyelid position, by processing in a manner of integrating a temporal change in speed and a temporal difference amount in position.

Here, because body movement is a slow motion, it can also be considered to eliminate the influence of that motion from a calculated temporal change in eyelid position by using a highpass filter or the like. The dotted line graph in FIG. 5 shows temporal changes in the eyelid position corrected by such a technique. By doing so, there is little problem on the detection of an eyelid movement of a width of approximately 10 mm such as with normal eye blinking, but the detection accuracy of a small eyelid movement on the order of 1 mm wide declines. That is, using a highpass filter produces a problem that an incomplete elimination of the influence of body motion prevents a separation from the remaining motion from being realized by only numerical analysis, in addition to a problem that the originally small eyelid movement is reduced in amplitude. In contrast, according to the correction method by the position correcting unit 15, position information from which the influence of an eye area movement has been removed can be efficiently obtained.

In addition, the position correcting unit 15 may be constructed so as to correct the influence of involuntary eye movement when calculating an eyelid position. Typically, involuntary eye movement includes tremor that is minute movement with a frequency of around 100 Hz and an amplitude on the order of 1 μm (20 to 40 second angle), drift that is a slowly drifting movement, and flick (also called microsaccade) that is saccadic eye movement (saccade) with 0.04 degree angle to 2 minute angle that occurs after drift. The position correcting unit 15 detects a temporal change in the speed of the subject's eye movement based on an image signal, extracts based thereon the period of flick being a rapid eye movement, and at timing where the eye speed in that period has exceeded a predetermined threshold, corrects the eyelid position so as to cancel out the influence of flick. Also, the position correcting unit 15 may process a temporal change in eyelid position using a frequency filter in order to eliminate the influence of drift movement.

The feature amount calculating unit 17 of the processor 9 calculates a feature amount regarding the subject's blinking based on a temporal change in eyelid position output from the position correcting unit 15, and then outputs the feature amount. For example, as the feature amount to be calculated, an eye blink feature amount is calculated that includes any of the average speed (eyelid closure average speed), maximum speed (eyelid closure maximum speed), eyelid closure distance, upper eyelid movement distance, and lower eyelid movement distance at the time of eyelid closure, eyelid closure rate, required time at the time of eyelid closure motion (eyelid closure period), eye closure period, average speed (eyelid opening average speed), maximum speed (eyelid opening maximum speed), required time at the time of eyelid opening motion (eyelid opening period), eyelid opening distance, upper eyelid movement distance, and lower eyelid movement distance at the time of eyelid opening, eye blinking frequency, blink-to-blink time interval, frequency of eye blinking to perform irregular motion, and frequency of specific eyelid movement. Then, the feature amount calculating unit 17 outputs an analysis result based on these feature amounts to an external device via a network. Parameters when analyzing an eye blink feature amount are statistics including a mean, a variance, a standard deviation, kurtosis, and skewness, a median, a quartile point, a maximum, a minimum, a mode, a maximum and minimum, etc., of the eye blink feature amount. Also, the feature amount calculating unit 17 may directly output an analysis result to an output means such as a display device or memory directly connected to the processor 9. As the analysis result, a result of a comparison with an internal database or a result of a comparison with an external database connected via a network may be output besides the feature amount itself, and data indicating a temporal change in eyelid position, data indicating a temporal change in eyelid speed, and data representing an image signal of a processing target may be output along therewith.

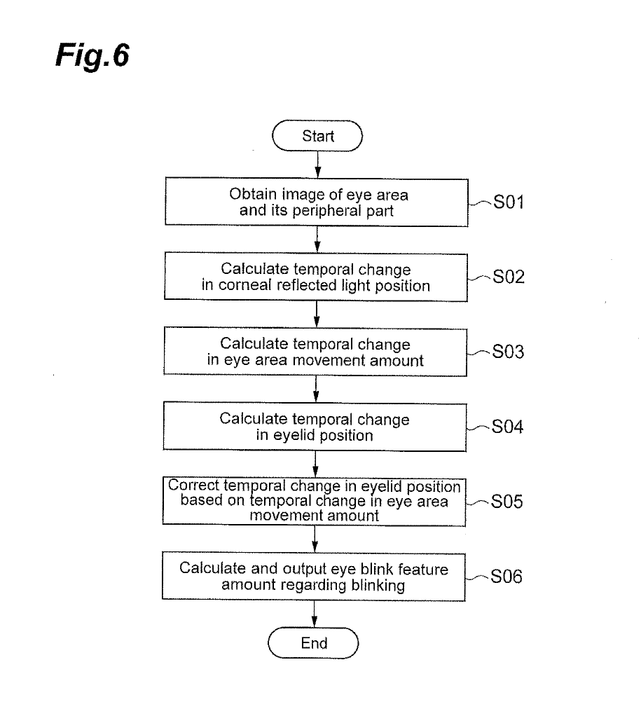

Next, description will be given of a detailed procedure of operation to calculate an eye blink feature amount by the eyeblink measurement system 1, while the eyeblink measurement method according to the present embodiment will be described in detail. FIG. 6 is a flowchart showing a procedure of operation to calculate an eye blink feature amount by the eyeblink measurement system 1.

First, referring to FIG. 6, when a measurement operation is started, an image signal of the part of the eye E including the eyelids is temporally successively obtained by the photodetector 7 (step S01). Next, by the movement amount calculating unit 13, a temporal change in the position of a corneal reflected light on the cornea of the subject's eye E is calculated based on the image signal (step S02). From that result, a temporal change in the movement amount of the subject's eye area is calculated by the movement amount calculating unit 13 (step S03). Thereafter, a temporal change in the subject's eyelid position is calculated by the position calculating unit 11 (step S04). Next, by the position correcting unit 15, the temporal change in eyelid position is corrected based on the temporal change in eye area movement amount (step S05). Then, by the feature amount calculating unit 17, an eye blink feature amount is calculated and output based on the temporal change in eyelid position after correction (step S06). Here, the processing from the calculation of a corneal reflected light position in step S02 to the correction in step S05 may be performed in real time during a measurement by the photodetector 7, or a part or the whole of the processing may be performed after the measurement. Also, the three steps S02, S03, and S04 following the image acquisition of the eye area and its peripheral part in S01 may be respectively independently performed, or may be simultaneously performed in parallel.

By the eyeblink measurement system 1 having been described above, an image signal of a reflected light from the part including the subject's eyelids and eye E is generated, a corneal reflected light position and an eyelid position in said part are calculated based on the image signal, and then the eyelid position is corrected based on the corneal reflected light position, and from a temporal change in the corrected eyelid position, a feature amount regarding blinking is calculated. This allows calculating a temporal change in eyelid position corresponding to a movement of the eyelid itself by a simple calculation even when the relative position of the subject's eye E with respect to the apparatus changes. As a result, a feature amount regarding blinking can be obtained at a high speed and high accuracy from the temporal change in the subject's eyelid position.

An eye blink feature amount can also be calculated from a difference image of images obtained by a video camera if it is a feature amount such as the eye blinking frequency or eye blinking period. However, it is difficult to stably obtain a feature amount such as the eyelid closure-time maximum speed or eyelid opening-time maximum speed, or a feature amount that quantifies the process of motions, such as an irregular motion so as to suspend an eye closure motion followed by resuming eyelid closure in a few milliseconds or shifting to an eyelid opening motion, an irregular motion at the time of eyelid opening, or eyelid motion of a minute eyelid movement. Such a problem leads to erroneous detection of an eyelid position and erroneous detection of an eyeblink motion to reduce the reliability of a quantitative analysis of blinking motion, which is a problem that affects the detection accuracy of a minute eyelid movement of about 1 mm that is observed in Parkinson's disease patients. By the present embodiment, also obtaining such a feature amount at a stable high accuracy is enabled.

Also, when the subject's gaze shifts, and for example, the gaze shifts upward during measurement, the shape of the eyelid present at a position to cover the eye changes, and the change tends to be erroneously detected as a minute eyelid movement. Also, there is a tendency when the subject's head moves that an eyelid position is erroneously detected as a result of the position of the eye area and skin part in a camera detection range changing, and as a result, blinking motion is also erroneously detected. By the eyeblink measurement system 1 of the present embodiment, such erroneous detection is prevented.

However, the present invention is not limited to the embodiment described above.

The eyeblink measurement system 1 described above may adopt the following construction as a construction to obtain a detection result of a corrected eyelid position.

For example, an imaging range of an image signal to be output from the photodetector 7 may be corrected based on a movement amount of the subject's eye area calculated by the processor 9. Such correction is performed by correction of the photographing angle of view of the photodetector 7, correction of the read-out position of an image signal, focus adjustment of the lens, adjustment in the relative position between the photodetector 7 and the subject, etc. The adjustment in the relative position between the photodetector 7 and the subject is realized by a construction of a camera main body and an optical system unit of the photodetector 7 being constructed by an identical unit and moving the unit. Also, a construction of driving an optical system unit including the lens may be adopted, or a construction may be adopted that is capable of adjusting the position of an image to be made onto the camera by using a MEMS mirror disposed in front of the camera or an element similar thereto. This allows obtaining an eyelid position corresponding to eyelid movement by adjusting the photodetector 7 without performing correction by image processing.

Also, a calculation target region for an eyelid position in an image signal may be corrected in the processor 9 based on a movement amount of the subject's eye area calculated by the processor 9. For example, in the processor 9, a calculation target region for an eyelid position is corrected by an image processing including position correction and expansion/reduction processing being applied to an image signal output from the photodetector 7 as a target. This allows obtaining an eyelid position corresponding to eyelid movement using a calculation target region adjusted in the range in an image signal without adjusting the photodetector 7.

In the above-mentioned embodiment, it is preferable that the feature amount includes a feature amount regarding an eyelid speed at the time of eye blinking. Having such a feature amount as a processing target allows obtaining a feature amount regarding a target person's eyelid speed at a high speed and high accuracy.

What is claimed is:

1. An eyeblink measurement method comprising:
detecting light from a part including a target person's eyelid and eye and outputting a detection signal of the light, using a photodetector;
calculating a position of a corneal reflected light produced on the eye in the part and a position of the eyelid in the part based on the detection signal;
correcting the position of the eyelid based on the position of the corneal reflected light; and
calculating a feature amount regarding blinking based on a temporal change in the corrected position of the eyelid.

2. The eyeblink measurement method according to claim 1, wherein the feature amount includes a feature amount regarding an eyelid speed at the time of eye blinking.

3. An eyeblink measurement apparatus comprising:
a photodetector configured to detect light from a part including a target person's eyelid and eye and output a detection signal; and
a processor electrically coupled to the photodetector and configured to input the detection signal, wherein
the processor is configured to
calculate a position of a corneal reflected light produced on the eye in the part and a position of the eyelid in the part based on the detection signal;
correct the position of the eyelid based on the position of the corneal reflected light; and
calculate a feature amount regarding blinking based on a temporal change in the corrected position of the eyelid.

4. The eyeblink measurement apparatus according to claim 3, wherein the feature amount includes a feature amount regarding an eyelid speed at the time of eye blinking.

5. A non-transitory computer-readable medium containing program instructions for causing a processor to perform the method of:
in an eyeblink measurement apparatus that measures eye blinking of a target person using an image of a part including the target person's eyelid and eye,
calculating a position of a corneal reflected light produced on the eye in the part and a position of the eyelid in the part based on the image;
correcting the position of the eyelid based on the position of the corneal reflected light; and
calculating a feature amount regarding blinking based on a temporal change in the corrected position of the eyelid.

6. The non-transitory computer-readable medium according to claim 5, wherein the feature amount includes a feature amount regarding an eyelid speed at the time of eye blinking.
